(19) United States
(12) Patent Application Publication
(10) Pub. No.: US 2017/0372018 A1
(43) Pub. Date: Dec. 28, 2017

(54) Melrose Pain Solutions® Method and Algorithm: Managing Pain in Opioid Dependent Patients

(71) Applicant: Melrose Pain Solutions LLC, Naples, FL (US)

(72) Inventors: Melanie Rosenblatt, Boca Raton, FL (US); Joseph Pergolizzi, Naples, FL (US); Liana McCormick, Hoboken, NJ (US)

(21) Appl. No.: 15/636,331

(22) Filed: Jun. 28, 2017

Related U.S. Application Data
(60) Provisional application No. 62/355,536, filed on Jun. 28, 2016.

(51) Int. Cl. G06F 19/00; A61B 5/00

(52) U.S. Cl. CPC G06F 19/325; G06F 19/3431; A61B 5/4824; A61B 5/4836; G06F 19/34

(57) ABSTRACT

The present invention provides a novel, comprehensive approach for the effective, safe and compassionate management of pain and opioid dependency, both in inpatient and outpatient settings, through the various stages of patient contact with the current healthcare system (e.g. initial encounter, treatment initiation, inpatient care, discharge, and post-discharge/chronic management) via innovative methods and treatment algorithms that provide consistent, repeatable and material advances in potential and high-risk, opioid-dependent patient management.

### FIELD OF THE INVENTION

The present invention provides a novel method and treatment algorithm for safe, effective, and consistent management of pain in the hospital and subsequent outpatient, setting. A key area of impact of the Melrose Pain Solution® (MPS) system is the treatment of the complex pain patient who uses and is dependent on high dose opioids. Currently there are no protocols that address the treatment and management of pain in complex, opioid dependent patients. These patients receive fragmented, inconsistent, and heterogeneous treatment leading to increased morbidity, mortality and cost.

### BACKGROUND

The treatment of pain (both acute and chronic) is becoming increasingly challenging. Even as the availability of existing prescription opioid medications ever increase and continue to proliferate the drug market, newly developed opioid medications are continuously injected into the drug pipeline, and the number of patients seeking relief is ever on the rise. In opposite, the ability to effectively control pain and ma attain the opioid utilizing patient pain population has steadily declined. The inevitable ramifications of failed modalities and therapies clearly have social, public health, economic, legal, and medical impacts.

Over 100 million Americans suffer from chronic pain at a cost of approximately $630 billion per year. The current treatment measures are often inadequate, fragmented, inconsistent, costly, and at times exacerbate the patient's condition. Recent efforts by the FDA to curtail opioid abuse through the rescheduling of certain hydrocodone containing products has further aggravated the pain patient plight by simply “shifting” abuse and misuse to other drugs, including illegal drugs. This results in more patients entering hospital systems and straining limited resources because of the inadequacy of current pain management and treatment options. Patients suffering from pain are often left with poor alternatives, break-through pain, inconsistent care, potential for harm, and increased healthcare costs. Plainly a paradigm shift is needed in treating pain, generally, and in correcting the natural consequences of this failed model, specifically.

While the United States is just under 5% of the total world population, it consumes up to 80% of the world opioids (indicting 99% of the world's supply of hydrocodone) through over 250 million prescriptions written annually. Equally, some 3.71 million e-prescription (out of 1.6 billion total), accounting for 307 million dosage units, were electronically transmitted for oxycodone and hydrocodone combined in 2016 according to Surescripts®. Consistently, hydrocodone is placed in the top ten most prescribed drugs, and is often the number one drug in several surveys resulting in a 24-billion-dollar market. Patients are often started on opioid medications for the treatment of acute, severe pain, which may progress to chronic pain and can lead to escalating dosing and opioid dependency. Even after the pain is controlled and the sequelae of injury or surgery have subsided, an appreciable number of-patients continue to use opioids for recreational, non-medical use. Sometimes these two groups, those experiencing pain and those not, create extensive overlap and are often indistinguishable. In the hospital setting both groups present similar challenges in pain management, thus augmenting the complexity of patient care.

According to the Centers for Disease Control and Prevention (CDC), opioids (including prescription pain relievers plus heroin) killed over 28,000 Americans in 2014 and more than half of those overdoses involved prescription medications (rather than heroin) leading lethal drug overdose to be the leading cause of accidental death in the U.S. In fact more overdose deaths can be attributed to prescription pain relievers than to heroin and cocaine combined. From 1999 to 2014, not only did over 165,000 Americans die of an overdose related to prescription pain relievers, in the same time period the number of opioid prescriptions quadrupled as well. Additionally, the latest data available in every state and the District of Columbia (a 2014 compilation and report by the Agency for Healthcare Research and Quality) shows 1.27 million emergency room visits or inpatient stays for opioid related issues in a single year (a 64 percent increase in inpatient admissions and a 99 percent increase for ER treatment since 2005). Thus, it can be deduced from the above, without much uncertainty, that where burgeoning demand meets indiscriminate opioid prescribing habits and increased access leads to increased usage, an unabated proliferation of addiction and dependence cannot help but to flourish.

In 2014, about 2 million Americans were either opioid dependent or abusing opioids. These numbers represent only the tip of the iceberg. Among Americans 12 years of age and older, 6.8 million reported the nonmedical use of a psychotherapeutic agent in the preceding month (data from 2012). In 2012, 335,000 Americans (0.1% of national population) reported using heroin that month. And while the actual picture of overdose deaths may contradict the prevailing images of “street drug users”. Sixty percent of opioid overdose deaths occur in the individuals taking opioids which have been prescribed according to the current guidelines (of which 20% are taking the so called “low-dose” opioid therapy of 100 mg/day morphine equivalents or less). Opioid-related death rates are higher for patients taking high-dose opioid therapy, but can occur at low doses as well.

Plainly, too, pain is an epidemic in and of itself and constitutes a true public health crisis. Over 100 million Americans suffer from some form of chronic pain, an aberrant maladaptive condition that can be debilitating, disabling and decreases quality of life. Opioids have gained increased entrance and acceptance into communities through prescribing and dispensing of, opioids to treat all levels of pain low, moderate and severe. Pain and associated resource consumption and expenses, including, but not limited to, lost productivity, may cost the United States more than $600 billion annually. Many of these patients are prescribed opioid pain relievers for long-term therapy, a practice described, in the literature and which is set forth based on guidelines, but one which remains controversial. Woefully, while pain control has been recognized as a fundamental human right, it is far too often under treated, treated inconsistently, or treated incorrectly all together. Compounding the issue, adequate analgesia is one of the subjects in the Hospital Consumer Assessment of Healthcare Providers and Systems (HCAHPS) hospital surveys that are designed to help patients evaluate their hospital care experience in the new value-based purchasing reimbursement model. Thus, physicians find themselves professionally and ethically obligated to treat pain, and, now face the constraints of protecting the financial interest of the hospitals (via HCAHPS survey scores) in which they serve.

The treatment of opioid-dependent individuals (which the literature sometimes refers to as “opioid addicts”) is not discussed as much or as frankly as the treatment of pain in the medical literature. The terminology used by experts to talk about opioid-dependent individuals has been fuzzy and sometimes even misleading—the literature favors terms like “inappropriate use,” “non-medical use,” “opioid misuse,” and “opioid abuse,” not to mention more descriptive terms like “chemical coping” and “Substance Use Disorder”.

An “opioid addict” is a straightforward term, but it encompasses a complex biopsychosocial phenomenon. The short definition of addiction by the American Society of Addiction Medicine emphasizes the complexities of addiction, “Addiction is a primary, chronic disease of brain reward, motivation, memory and related circuitry. Dysfunction in these circuits leads to characteristic biological, psychological, social and spiritual manifestations. This is reflected in an individual pathologically pursuing reward and/or relief by substance use and other behaviors. Addiction is characterized by the inability to consistently abstain, impairment in behavioral control, craving, diminished recognition of significant problems with one's behaviors and interpersonal relationships, and a dysfunctional emotional response. Like other chronic diseases, addiction often involves cycles of relapse and remission. Without treatment or engagement in recovery activities, addiction is progressive and can result in disability or premature death.”

Further clouding this area, some patients are clearly and exclusively pain patients or drug addicts, but there is a considerable overlap between the true patient suffering from pain and the chemically addicted individual. Manifestly, it is Incumbent on clinicians, the healthcare system, policymakers, and the public alike to understand and recognize that a person may have a legitimate pain indication for opioids and still be opioid dependent.

It is nearly inevitable that opioid dependent patients, including addicts, will eventually come through the hospital system, sometimes seeking emergency pain control, rescue for overdose, treatments or procedures related to their addiction (for example, an abscess at the injection site), or other related reasons. When they enter the healthcare system, treatment of their pain is the first priority. Current guidelines and modem practice encourage physicians to treat pain effectively and promptly. Moreover, today's taxed and hectic healthcare environment requires most physicians to see many patients in a single day and make clinical care decisions quickly. Clinicians often do not have the time or proper training to recognize drug-seeking behavior—particularly given the fact that drug-seeking patients are known to be exceptionally adept at concealing their true motivations.

Thus, there is a significant, well-recognized and unmet need in the art for methods and systems that address both pain control and opioid addiction and dependence in a reliable, consistent, safe and effective way. The present invention satisfies this long-standing need in the art.

### BRIEF DESCRIPTION OF THE FIGURES

FIG. 1 depicts a Venn diagram representing the relationship between and among patients using opioid medications to treat pain and those that continue to use opioid medications for recreational, non-medical use.

FIG. 2A depicts a representative decision flowchart for Melrose Fain Solutions® in which certain types of information is gathered, stored, analyzed and processed in accordance with the present invention.

FIG. 2B depicts additional information which is incorporated into the decision-making process as an adjunct to the information in FIG. 2A which is gathered, stored, analyzed and processed in accordance with the present invention.

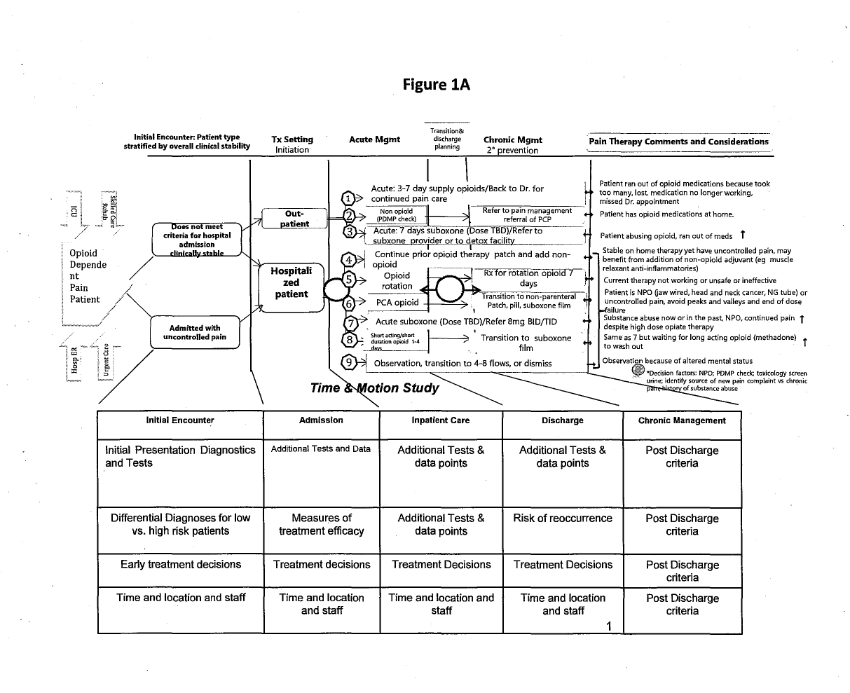

Still other objects and advantages of preferred embodiments of the present invention will become readily apparent to those skilled in this art from the following detailed description, wherein there is described certain preferred embodiments of the invention, and examples for illustrative purposes.

### DESCRIPTION OF PREFERRED EMBODIMENTS

Advantages of the present invention will become readily apparent to those skilled in the art from the following detailed description, wherein there is described certain preferred embodiments of the invention, and examples for illustrative purposes. Although the following detailed description contains many specific details for the purposes of illustration, one of ordinary skill in the art will appreciate that many variations and alterations to the following details are within the scope of the invention. Accordingly, the following embodiments of the invention are set forth without any loss of generality to, and without imposing limitations upon, the claimed invention. While embodiments are described in connection with the description herein, there is no intent to limit the scope to the embodiments disclosed herein. On the contrary, the intent is to cover all alternatives, modifications, and equivalents.

As used herein, the terms “comprising,” “having,” and “including” are synonymous, unless the context dictates otherwise.

According to one preferred embodiment, the present invention provides well-tested methods for managing and treating pain patients, creates a lattice and framework for consistent, repeatable techniques and methods for effective pain control, and addresses the public health care crisis of pain management and opioid dependence. The system and methods of the present invention have numerous benefits. For example, the present invention will fill a significant void for patients suffering with pain in need of acute medical care (including those patients who are and are not also patients with substance abuse issues), patients on high dose opiates with unrelieved and persistent severe pain, patients with frequent admissions of uncontrolled pain, and patients experiencing drug overdose. The present invention will also improve patient satisfaction and quality of life (thereby enhancing HCAPS scores), decrease the burden on an already strained healthcare system, enhance recognition and appreciation of the interrelation of pain and addiction, and, ultimately, help to generate significant healthcare savings (in the millions of dollars); moreover, the present invention will also improve healthcare worker satisfaction through increased reliance upon a uniform, established protocol, lessen after-hour phone calls (e.g., to doctors, charge nurses, and administration), reduce frequent hospital admissions and readmissions, improve remission rates, and reduce untoward harm events.

The present invention also provides methods for healthcare professionals to address both pain control, and opioid addiction in a consistent, reliable, safe and effective way. Upon observing that opioid addicts can be pain patients and, conversely, pain patients can be opioid addicts, implementation of the present invention provides the surprising and unexpected benefits of providing safe, effective, reliable and consistent pain control to everyone who needs it, without enabling opioid addiction. The present invention provides significant benefits to high-risk individuals and offers real-world pragmatic solutions to our ongoing public and healthcare crisis.

As used herein, a potential “high-risk individual” is classified by the Melrose Pain Solutions® system as a patient who meets identifying criteria according to the table below:

| Any One of the Following | Any Two or More of the Following |
|---|---|
| Acknowledges substance | History of incarceration |
| abuse | |
| IV Drug abuse | History of high dose opiate, history |
| | illicit substance use, history of DUI |
| Alcoholism | Doctor shopping, drug diversion |
| Reasons for admission | Family reporting of drug use |
| drug overdose | |
| Reason for admission | Disruptive behavior, non-compliance |
| altered mental state, lethargy | |
| Transfer from drug | History of drug treatment, discrepancies |
| treatment center | in story |
| Positive urine toxicology | Asking for opioid drugs by name, and |
| screen for illicit substance | by specific route of administration |
| | Frequent hospital admissions |
| | Ante-cubital spider bite |
| | Cellulitis, infective endocarditis, |
| | osteomyelitis of the spine, Hepatitis C |

In a preferred embodiment, the methods and system of the present invention significantly help to manage and treat patients who seek pain relief. One such preferred approach contemplated by the present invention is called the “Melrose Pain Solutions®” system which is used to manage and treat patients who seek pain relief. The “Melrose Fain Solutions®” system can be effectively utilized in many settings, including, but not limited to, an acute-care hospital, emergency department, long-term care residence, clinic, and/or physician's office. It is preferred that the “Melrose Pain Solutions®” system is taught and practiced by all members of a healthcare community, broadly, and a healthcare team, specifically, for maximum efficacy and impact.

It is also contemplated that the “Melrose Pain Solutions®” system can be made available to healthcare practitioners, for instance, via a “mobile app” or other type of software application, or via any other electronic or digital means, which can be implemented on one or more hardware devices such as computer, smartphone, tablet, or any other suitable electronic or computerized device. In one embodiment, the “Melrose Pain Solutions®” system is implemented as a secure, confidential, interactive, computerized system, which has an easy-to-use interface, that utilizes one or more decision-assisting algorithms, which may be implemented as an application (e.g. a mobile application or software application) running on a computer system, further wherein the application may be operated using computer hardware, including a computer processor. The interactive, computerized system gathers and processes information regarding a patient, and uses this information to assist a healthcare professional with identifying and determining optimized management and treatment protocols for individual patients. The Melrose Pain Solutions® system can also be used for rural tele-medicine in undeserved areas. The “Melrose Pain Solutions®” system can be operated using any computer platform, wireless platform or other electronic platform (such as a smartphone, tablet, laptop, robot or other similar device), thus allowing the healthcare practitioner to gather, analyze, utilize, store and retrieve information, for instance, about the status of a particular patient or other at-risk individual, and assist in identifying and determining optimized management and treatment protocols for individual patients. The secure, confidential, interactive system can preferably contain data and information about several individual patients and can be implemented in any hospital, clinic, doctor's office or other healthcare facility. Access to the secure, confidential, interactive system can also be made available after payment of a fee, for instance, a fee paid by the hospital, clinic, doctor's office, other healthcare facility, or insurance company.

In a preferred embodiment, a healthcare practitioner can preferably access the “Melrose Pain Solutions®” interactive system of the present invention, for example, may be accessed by a secure website (which is password and/or encryption protected) via a personal computer or PC, or via access to any other type of computer terminal network terminal, and/or other electronic device, including but not limited to a laptop, tablet, robot or smartphone. The computer or other electronic device can be operated using any type of operating system including but not limited to, for example, any type of Linux®, Apple®, Android® or Windows® brand operating system. In preferred embodiments, the computer or other electronic device has a screen and a keyboard and the keyboard can, for example, be a physical keyboard, an onscreen virtual keyboard, or a “touch-screen keyboard” (e.g. a keyboard that is accessed via touching the screen). The screen can also be a “touch screen” which allows the user to use the interactive system by touching the screen with either their fingers, a stylus, or by other means. The user can also preferably zoom in or zoom out to change the size of the content when they are viewing the content via the interactive system.

By way of non-limiting example, the interactive system can include any number of hardware and software components that together provides a secure and reliable system which is operable for providing users with access to the “Melrose Pain Solution®” system. By way of non-limiting example, hardware components can include, but are not limited to, a monitor, keyboard, hard disk drive, sound card, graphic cards, memory (RAM), motherboard, and computer data storage. The interactive system can also optionally include one or more speakers, and accompanying hardware and software components that allow a user to listen to audible components from a file. The interactive system can also optionally include a microphone and accompanying hardware and software components that allow a user to record his or her own audio input which, for instance, can be transcribed and allow a healthcare practitioner to contribute additional information, e.g., regarding a patient's status.

More preferably, a user of the “Melrose Pain Solutions®” system can securely and confidentially store data aid files, via password and/or encryption protection, including for instance files regarding a patient's status, on one or more remote data servers that can be accessed by other healthcare professionals confidentially and securely. A user of the system can also preferably use one or more secure and customized web-based applications, for instance any suitable SaaS or “Software as a Service” application, to organize the data and files. A “cloud server” can also be utilized to store the files available on the interactive system, such as video files, patient records, graphics, images, etc, using any suitable cloud computing server architecture. These and other data-backup, server and storage technologies can be utilized in accordance with the present invention, such that healthcare professionals and authorized users of the interactive system can safely and reliably upload any type of audio and video content, and other data and files to a server, such as a network server or cloud-based server.

According to preferred embodiments of the present invention, the “Melrose Pain Solutions®” system of the present invention, as described herein, including any mobile application, software application, and/or customized Interactive system, and which can be utilized by healthcare professionals and authorized users, is preferably comprised of several components or “subsystems” which together reliably enables a healthcare professional to make informed clinical decisions about how best to treat a particular individual. These “subsystems” together serve to gather a great deal of information, e.g. about a particular patient, so that the best management and treatment decisions can be made. In such a manner, the “Melrose Pain Solutions®” system of the present invention allows for reliable, effective and efficient methods for identification of patients, methods for managing patients, and methods for treating patients. Collection of data from different patients (e.g. including patients of different age, gender, ethnicity, prescription records, health histories, etc) can also be compiled into a large, confidential, secure database, in such a manner that healthcare professionals can then acquire a larger data set, thus providing a very valuable database of information for analysis that will allow for an even better understanding of the patient population and provide for even better methods of identifying, managing and treating patients.

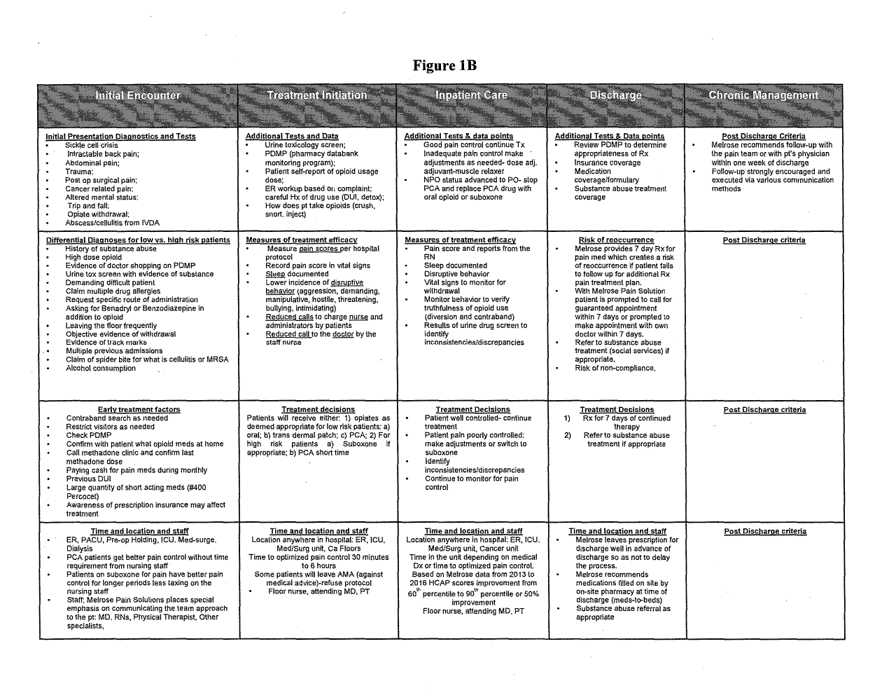

In accordance with a preferred embodiment of the present invention, a representative approach is depicted schematically in FIGS. 2A and 2B, in which opioid-dependent pain patients are identified and evaluated, and recommendations are made for management of the patients. Referring to FIG. 2A, a representative decision algorithm flowchart is shown in which certain types of information can be gathered, stored, analyzed and processed in accordance with the present invention. This information can, for instance, be stored in an Interactive system and made accessible via a mobile application or other software application, as described herein, and then used by healthcare professionals in a password protected, confidential and secure manner for better identification, management and treatment of patients.

Referring again to FIG. 2A, a potential opioid-dependent pain patient is initially identified by a healthcare professional, e.g. after being seen by the healthcare professional in an urgent care center, a hospital emergency room (ER), intensive care unit (ICU), skilled care rehabilitation center, or other healthcare setting. The patient is then categorized as falling within the guidelines of opioid dependency or outside of the guidelines of opioid dependency. If a patient is designated as falling under the guidelines of opioid dependency, the patient is then accessed on clinical stability, e.g., as an outpatient if not meeting criteria for hospital admission, or as a patient meeting the criteria for hospitalization. Referring to both FIGS. 2A and 2B, multiple types of information can be gathered based on an initial encounter with a patient. Various types of “Diagnostics and Tests” can be performed during the “Initial Encounter” with the patient. Examples of certain diagnostics and tests that can be performed during the “Initial Encounter” are shown in FIGS. 2A and 2B. The results of these Diagnostics and Tests can be entered into an interactive, dynamic system, for instance the “Melrose Pain Solutions®” interactive system, as described herein. During the “Initial Encounter” with the patient, “Differential Diagnoses for Low vs. High Risk Patients” can then be performed, and the information gathered from this “Differential Diagnoses for Low vs. High Risk Patients” can also be entered into the same interactive, dynamic system. One or more “early treatment factors” can then also be analyzed and the information obtained can also be entered into the same interactive, dynamic system.

After the Initial Encounter with the patient, treatment initiation begins, e.g. depending in part on whether the patient is deemed an opioid-dependent outpatient or a more critical opioid dependent hospitalized patient. This stage is referred to as the “Admission” stage (as shown in FIG. 2A) or “Treatment Initiation” stage (as shown in FIG. 2B). During this treatment initiation stage, additional information is gathered from additional tests (e.g. urine toxicology screen, PDMP (Prescription Drug Monitoring Program), etc.). This additional information is also entered into the interactive system, for instance the “Melrose Pain Solutions®” interactive system, as described herein. Moreover, information regarding “Measures of Treatment Efficacy” (examples of these measures are shown in FIG. 2B) is also gathered and this additional information is also entered into the same interactive, dynamic system, for instance the “Melrose Pain Solutions®” interactive system, as described herein. Significantly, during the “Admission” stage (as shown in FIG. 2A) or “Treatment Initiation” stage (as shown in FIG. 2B), information regarding a patient's “Treatment Decisions” is also entered into the same interactive system.

Referring again to FIGS. 2A and 2B, additional information can be gathered about a patient during the “Inpatient Care” stage. Including information from additional tests, measures of treatment efficacy, and information regarding treatment decisions. Referring to FIG. 2A, information that has been collected about a specific patient can be utilized to make very specific recommendations or decisions about acute management or chronic management of a patient. Representative examples of steps that may be taken for acute management or chronic management of a patient are shown in FIG. 2A. Treatment with Suboxone®, for instance, using a Suboxone® film or tablet (or a similar buprenorphine and naloxone combination), is one example of a step that may be taken for acute management or chronic management of a patient. In like manner, referring again to FIGS. 2A and 2B, additional information can be gathered about a patient during the “Discharge” stage and “Chronic Management” stage. One representative and preferred implementation of the system, as depicted schematically in FIGS. 2A and 2B, and as described in more detail herein, is a “Melrose Pain Solutions®” system. All additional information can likewise be entered into the interactive system, for instance the “Melrose Pain Solutions®” interactive system, as described herein. As further described herein, the “Melrose pain solutions®” system can be made available to healthcare practitioners, for instance, via a “mobile app” or other type of software application, or via any other electronic or digital means, and implemented on one or more hardware devices such as computer, smartphone, tablet, robot, or any other suitable electronic or computerized device.

In one embodiment, the “Melrose Pain Solutions®” system is implemented as a secure, confidential, interactive, computerized system which has an easy-to-use interface, that utilizes one or more certain decision-making algorithms, and which may be implemented as an application (e.g. a mobile application or software application) running on a computer system, further wherein the application may be operated using computer hardware, including a computer processor capable of securely safeguarding protected patient information (PPI). The interactive, computerized system gathers and processes information regarding a patient, and uses this Information to assist a healthcare professional with identifying and determining optimized and customized management and treatment protocols for individual patients.

The interactive, computerized system of the present invention has a number of additional and significant advantages, with regard to assisting a healthcare professional with identifying and determining optimized and customized management and treatment protocols for individual patients, e.g. optimized methods for managing and treating wide range of pain patients. As further described herein, the interactive system of the present invention can significantly help healthcare professionals with the process of effectively managing and treating a wide variety of patients in a customized and consistent way.

### Identifying Potential Opioid Dependent Patients

Patients enter the hospital or other portal into the healthcare system ostensibly seeking pain relief. Yet, at times, the situation is more complicated because there may be an additional motivation: a genuine need for analgesia, opioid seeking behavior, or seeking relief from withdrawal symptoms. Addressing these complicated situations requires a reliable, consistent, and structured method, in accordance with the present invention, the “Melrose Pain Solution®” system helps insure that all the pain patients receive appropriate pain management, while not necessarily acquiescing to their demands. While the treatment of pain does not discriminate depending on history or behavior. Identifying potential high-risk patients can be useful in helping to predict and proactively address behaviors and drug-seeking tactics. This stratification shapes how the physician and healthcare team may, in combination with sound professional judgment, handle inpatient and aftercare.

High risk patients may reveal themselves in any numbers of behaviors, questions they ask, and requests they make. They may frequently request specific drugs by name or exhibit detailed knowledge about pain medication such as dosing regimens and specific route of administration. Sometimes they already have prescription opioids but request higher doses or different agents. They frequently offer reasons as to why they want a specific drug and why other pain relievers are not appropriate for them (“It doesn't work for me” or “I'm allergic”). Patients at risk frequently want specific opiate drugs plus benzodiazepines, IV Benadryl and deflect attempts to control their pain with other agents or treatments.

The “Melrose Pain Solutions®” system of the present invention can be used to obtain more relevant insights faster (a material benefit under the conditions of limited time and resources). Using the “Melrose Pain Solutions®” approach, the patients are asked a series of structured questions that seek to access relevant, consistent, and necessary information from the patient in different ways. (The intent is to identify the nature, location, and cause of the patient's pain and to ascertain if the patient is currently taking prescribed, non-prescribed, or illicit drugs.) The potential high-risk patient often tries to conceal his/her addiction, but repetitive questions that approach the same topics from various angles can often break through the façade. The healthcare professional should ask structured screening questions designed to reveal prior drug use, alcohol use, family life dysfunction, arrests for drug use, previous rehabilitation efforts, and family history of substance abuse (among other questions). A subset of opioid addicts may be considered high functioning, that is, they may hold down jobs, maintain a household, and have intact personal relationships. Often the burden of addiction takes its toll to the point that they are unable to function normally for protracted periods of time, if at all. The intent of the structured Melrose Pain Solutions® system is not to intimidate or shame the patient or make the patient defensive, but rather to get a more holistic picture of the patient and to avoid making assumptions. Certainly, a poor work history and chronic pain does not mean a person is an addict. The clinician needs to get a true picture of the patient's life, true nature of the pain, and a complete history of opioid use. This stage is for fact-finding and correct stratification of the patient, while seeking to decrease the number of false positive patients. See Table 1.

Table 1 shows representative questions and techniques for the “Melrose Pain Solutions®” methods and systems of the present invention as described herein. These questions listed herein in Table 1 are representative samples and may be modified to meat the needs of the healthcare professional and the patient.

TABLE 1
Techniques for the Healthcare Professional
Ask questions politely but firmly, and be persistent; Approaching the
subject from different angles helps overcome obfuscation.
Be systematic in the interview; don't abandon the line of questioning even
if, after the initial questions, the patient appears irritated or uncooperative.
If active drug use is suspected, a contraband search should he conducted.
Ask questions in a methodical consistent manner, rephrasing a question
if a discrepancy is identified.
If possible or appropriate, continue probing questions after the initial
interview.
Sample Questions that a Healthcare Professional can ask a Patient
Do you normally take pain medicines at home? How do you take it?
Crush it? Snort it? Inject it?
Have you ever used drugs in the past? When you were younger?
Do you use marijuana?
Do you drink alcohol? How much? How Often?
Have you ever had a DUI?
Have you ever been in rehab?
Have you ever been arrested for anything drug related?
Do you work?
What kind of work do you do?
Who do you live with?
Do you have any children? Do they live with you?
What surgeries have you had in the past?
Has anyone in your family been in rehab before?
Have you ever been arrested for drugs before?
Do you drink?
Have you ever been arrested?
Do you smoke?
Have any medications worked better for you than others?
Have you ever tried on Suboxone ® or methadone or the like?
Why are you asking for this specific opioid? Have you had it before?
Do you drive to work?

Is a preferred embodiment, there is a method to manage and treat patients who seek pain relief. This method comprises identifying the patient asking questions of the patient, prescribing the appropriate arid safe pain treatment (i.e. not necessarily the drag of choice), offering a realistic plan with the ultimate goal of arriving as early as possible in the course of treatment at a treatment plan that can be continued in an outpatient setting. In another embodiment, high risk patients are identified through a series of structured questions and as the hospital treatment progresses more actionable information becomes available.

High risk patients are often very skillful in denying or at least minimizing their addiction, possibly believing they are exceptional and “can handle it” while others cannot. Despite the persuasiveness, the healthcare professional, should ask questions systematically and address any inconsistencies. When the patient contradicts himself/herself, the clinician should realize this was information that should be clarified. If the patient becomes hostile or defensive a good approach is to softly explain that the questions help in identifying the best treatment path. If necessary, step away and return with a clinical colleague.

### Confronting the Addict

High risk patients may demand specific medications, formulations, or doses, and may resort to disruptive tactics (outbursts, tantrums, negotiations, arguments, rage, threats, flirtation, or persuasion) to try to convince the prescriber to do what they want. Addicts who realize they are not going to get the drugs they want may walk out. It is interesting to note that in no other area of medicine are prescribes faced with such persistent “patient negotiations”. For example, in infections medicine, the physician will discuss the patient's condition, may offer a few treatment options, and then prescribes the appropriate pharmacological regimen without having the patient demand or insist on antibiotic X instead of antibiotic Y or get angry if oral antibiotics are administered instead of IV antibiotics. Yet the healthcare system has come to expect and accommodate such demands from patients seeking pain control.

In yet another embodiment, the step of confronting the high-risk patient comprises discussing the patient's medical condition causing the pain and describing the pain control regimen, if appropriate. In some cases, the patient's pain may be managed with a non-opioid pain reliever, but if an opioid is required, the healthcare provider may prescribe buprenophine or buprenorphine/naloxone. This is the same approach, for a patient who is opioid naïve or opioid dependent. Many experienced drug users will decline Suboxone® (imprenorphine/naloxone), for example, claiming allergy or a “bad” experience. This step is important because it highlights their ambivalence to change and refusal of alternatives. Many will leave against medical advice (AMA). While this step might interfere with treatment of their medical condition, the Melrose Pain Solution® system has the distinct advantage that it does not enable addictive behaviors. In fact, MPS creates an opportunity for the drug addict to begin medication assisted treatment with Suboxone® which has been shown to help not only with their disease of addiction but with their pain as well.

High risk patients who are hospitalized or in long-term care settings may try to find enablers to bring or sell them drugs. In this scenario, the “Melrose Pain Solutions®” methods and systems of the present invention advocates restricting visitors. Contraband search may be warranted. In this situation, some addict patients will attempt to get around the regulations, persuade clinicians to give them special treatment, or attempt to leave the facility (which may not always be possible). In the event a patient does leave the facility even against medical advice, this is not necessarily a bad outcome as the patient was offered appropriate treatment by healthcare professionals which he/she refused, yet in the end the healthcare team did not enable the addiction.

Sometimes patients who already have prescriptions tor opioid medications will see a physician or visit the emergency room demanding more or different opioids. There are two main motivations for this patient. First, either the patient is frustrated over inadequate pain control or the patient is an addict seeking more and/or better drugs. The prescriber should first confirm the patient's current opioid regimen, who prescribed it, and how long the patient has taken it. The patient should be asked when the medication was last taken and what the dosing schedule is. Then the prescriber should assess the pain, using a 10-point scale where 0 is no pain at all and 10 is the worst possible pain imaginable. Many addicted patients will report very severe pain (10/10 is not unusual). At this point, the prescriber should use this information as a “teachable moment” If the patient is taking prescription opioids in moderate to large doses as directed and his or her pain is virtually unaffected by the drug, then clearly the opioids are not working. The patient will scramble to explain that the pain is getting worse or some new condition has intervened. The prescriber should then explain to the patient that the medication appears not to be working likely due to tolerance and to excessive high dose prior to admission. In accordance with the “Melrose Pain Solutions®” methods and systems of the present invention, the prescriber should take the opportunity to contact the patient's original physician and report the incident, that is, that the patient is soliciting more opioid analgesics from another physician. One notable shortfall of the healthcare system is that high risk patients are able to consistently exploit, is the fact that prescribers and other healthcare professionals do not usually make that phone call. Improved communications among prescribes in a community can help to prevent such patients from abusing the system.

### Prescribe a Pain Management Plan (Not Simply the Drug of Choice)

The “Melrose Pain Solutions®” (or “MPS”) approach of the present invention recognizes that physicians must treat pain. Many severe patients have painful conditions for which opioids might appropriately be prescribed. However, it is not up to the patient to select the drugs he or she wants; it is a physician's choice to prescribe responsibly. Healthcare providers should prescribe like the healthcare professionals they are, and not acquiesce to the patient's demands.

The “MPS” model of the present invention recognizes that buprenorphine (Belbuca®, Bunavail®, and Butrans®), is an outstanding analgesic product for a wide range of patients, particularly but not exclusively for addicts with pain indications. While buprenorphine can still be abused, its abuse potential is lower than other opioids. Buprenorphine is well known for its ceiling effect on respiratory depression. It has been shown in numerous clinical trials to be safe and effective against many types of pain. It is available in many formulations, including a transdermal patch, which allows for dosing and administration versatility.

Of coarse, not all patients in pain respire opioid analgesics. In some cases, it is appropriate to prescribe non-opioid agents, such as acetaminophen or nonsteroidal anti-inflammatory drugs (NSAIDs). These agents may be supplemented by muscle relaxants, antidepressants, or anticonvulsants to address other components of the patient's pain. Nonpharmacological options may be appropriate for some patients such, as physical therapy, hot or cold therapy, TENS (Transcutaneous Electrical Nerve Stimulation) units, or massage therapy. Combination approaches may also provide greater relief.

The urgency to avoid withdrawal symptoms (“dope sickness”) can be particularly intense and may be the driver behind the patient's insistence on getting more opioids fast. Buprenorphine will prevent withdrawal symptoms as well as provide pain relief. In fact, buprenorphine can “turn off” withdrawal symptoms for the patient which can interrupt the drug-seeking behaviors.

### Offer a Realistic Alternative

In accordance with the “MPS” methods and systems of the present invention, it is recognized that some addicted patients will leave the hospital setting if they do not get the drugs they want. This can be upsetting to some clinicians, but it is not necessarily a negative outcome. Far worse would it be if addicts came to a clinic and got exactly what they wanted.

While society finds it preferable that all addicts get treatment, it is not realistic to expect every addict to agree to the recommended intervention. Some will balk, and with varying degrees of intensity and anger. Others will enthusiastically embrace the treatment. Some will leave one emergency room and head for another. Yet if all hospitals across the nation embraced the MPS treatment model, and methods and systems of the present invention, there would be no other more “helpful” emergency room to visit. While the MPS treatment model, and methods and systems of the present invention, can work in an individual hospital or clinical setting, it has the potential to change the face of addiction across the nation if it was to be embraced as a comprehensive national program.

The Melrose pain solutions® model requires that the entire healthcare team be trained and educated in this treatment paradigm. Some patients can be particularly adept in figuring out who is the “weak link” in a system and might take advantage of the healthcare systems propensity to achieve patient satisfaction. Melrose Pain Solutions® system and method requires all healthcare providers in the system to not only be knowledgeable and follow the same protocol, they must also be professional, kind, patient, approachable, and compassionate. The patient should not be allowed to dictate his/her own care.

Many high-risk patients are characteristically unable to appreciate the depth or extent of their own problems. They may deny their drug use or trivialize it as a minor quirk. For that reason, many patients do not want treatment for their addiction, even when it is offered to them, or—at best—are ambivalent about beginning treatment, putting it off to some vague point in the future. The interview format works well in this setting.

At this phase of the treatment algorithm MPS system and method utilizes well established and previously described motivational interviewing techniques. The clinician should ask the patient: On a scale of 0 to 10 where 0 is not at all and 10 is the most likely, how willing are you to make a change? Most patients will answer with a rating of 3 or 4; they most likely will not say 0, but they may make a point to let the healthcare team know that they are not seriously looking for rehabilitation. At this point, the clinician should answer by saying, “Why so high? I would have thought you were going to say zero. Why a 3 or 4 aid not a 0?” This strategy forces the patient to argue in favor of making a change. In this setting, the patient may reveal to the physician some genuine concerns that can help give the prescriber greater insight into the patient. For example, some patients may report that they want to get custody of their children, hold down a job, save some money, or find a better place to live. Some will say simply they just want to have a “normal life.” The clinician should use these answers to encourage the patient to agree to better alternatives.

### Continue to Treat the Patient

For patients treated with buprenorphine, a transition in attitude occurs after a few days. These patients describe effective pain control and no withdrawal symptoms. Formerly difficult and demanding patients often regain their equilibrium and report to the healthcare team that they are feeling well. They are likely now to agree to remain on buprenorphine as an outpatient.

This work takes proper training, resources, and consistency within the healthcare system.

### The Role of Buprenorphine in the “Melrose Pain Solutions®” Methods and Systems of the Present Invention

In accordance with the Melrose Pain Solutions® system and methods of the present invention, it is recommended that clinicians prescribe buprenorphine, when an opioid is indicated, to treat pain in both pain patients and drug-seeking patients with painful conditions. Buprenorphine is a potent opioid, effective analgesic, and has a low abuse liability. It owes some of these characteristics to its unique pharmacology. Various forms of buprenorphine are available on the market in various delivery systems and can be used tor pain. Numerous clinical studies have found buprenorphine to be an effective pain reliever and it treats neuropathic pain and a broader array of pain phenotypes than do certain other opioids. Buprenorphine is associated with fewer side effects, notably less constipation, less cognitive impairment and it does not prolong the QT-interval of the heart. Buprenorphine is not immunosuppressive (as are morphine and fentanyl) and does not cause hypogonadism or adverse effects on the hypothalamic-pituitary-adrenal axis. It is recognized as one of the safest opioids to use for patients with compromised renal function. Finally, as mentioned earlier, it has a ceiling effect on respiratory depression, a potentially fatal adverse event associated with other strong opioids.

In accordance with the “Melrose Pain Solutions®” methods and systems of the present invention, buprenophine can be administered as a parenteral injection, a sublingual tablet, sublingual/buccal film, and a transdermal delivery system. These various formulations and doses allow for prescribing versatility. Furthermore, buprenorphine is an established treatment for opioid addiction with considerable evidence in the literature for its safety and efficacy in this setting.

### The Status Quo Versus the “Melrose Pain Solutions®” Method of the Present Invention

Chrome pain remains under-treated, opioid addiction has reached epidemic proportions, and most healthcare professionals are left in a quandary as to how to treat pain without fueling the opioid epidemic. Prescribers are expected to treat legitimate painful conditions in patients with active substance abuse. Patients have a right to expect appropriate pain management even under high risk circumstances.

Opioid addiction has become so prevalent that legal and political forces have become involved. The problem is vast and growing, and Melrose Pain Solutions® system offers a safe, effective, reliable, and consistent solution. If implemented broadly Melrose Pain Solutions® system has the potential to manage and solve the opioid epidemic by treating the high-risk patient each time they interface with the healthcare system.

The “Melrose Pain Solutions®” approach of the present invention places the focus an fighting addiction where it belongs: in the healthcare setting. Addicts frequently interface with the healthcare system—in fact, over time, it is almost impossible for a long-term drug addict to avoid hospitalization and frequent doctor appointments. The “Melrose Pain Solutions®” method and approach does not require some sort of outreach campaign or other efforts to find addicts, nor does it expect addicts to knock on the doors of treatment centers. It is the nature of opioid dependency that the patients—sooner or later and usually repeatedly—enter the healthcare system. It is the healthcare system that must be prepared to treat them. The “Melrose Pain Solutions®” approach systematizes this care and tenders it safe, effective, reliable and consistent.

If all hospitals and clinics and healthcare providers across America embraced the “Melrose Pain Solutions®” system and approach, addicts would not be able to demand their drug of choice from the healthcare system. They would get appropriate pain treatment and an opioid product that would prevent them torn going into withdrawal and they would get a frank discussion about their condition along with long term options for treatment. These are potential victories in our public health wars on two fronts—a victory for pain patients in that they get pain control and a victory for reducing opioid abuse in that opioids are not so freely dispensed thereby supporting continued addictive behavior.

Many people with addiction issues would like to overcome their dependence but just do not know where to turn. The “Melrose Pain Solutions®” model of the present invention recognizes that in the real-world clinical setting, many people with dependence issues may deny their addiction (at least at first) and, even if they begrudgingly admit some degree of drug dependence, often refuse help or approach options presented by caregivers with great skepticism. Most active addicts do not seek treatment on their own and may reject treatment when offered.

Overburdened hospitals and clinics end up providing addicted patients with the drugs they seek in an effort to placate patients and move them quickly through the system. In other words, addressing the real issue of addiction is trumped by the immediate goal of rapid patient throughput and limited resources. There are even some healthcare professionals who misplace their sympathy and think, it is helpful to at least ease the temporary suffering of an addict in pain by giving in to a request for a specific opioid. Still other healthcare professionals think, like the addict, that it is not such a big deal to provide an addicted patient with a few extra pills. The status quo is a system that demands a quick fix for pain, i.e. more opioids. The opioid epidemic has been fueled by a system that demands it.

There is therefore a significant and urgent need for better solutions and tools vis-á-vis the status quo. The Melrose Pain Solutions® system is the effective, safe, reliable, and consistent method and tool to treat pain in difficult patients. The present invention also accomplishes numerous objectives, including but not limited to the following:
Helps healthcare professionals to better understand and evaluate the current continuum of care of opioid dependent patients with pain in the hospital setting (From Initial Evaluation to Discharge)
Helps healthcare professionals to better understand and evaluate the current alternative decision pathways for the treatment of the opioid dependent patient with pain
Helps healthcare professionals to develop a workable model to address a broader public health care crisis
The present invention will fill a void for patients suffering with pain, including but not limited to these types of patients:
   1. Patients with substance abuse in need of acute medical care
   2. Patients on high dose opiates with unrelieved and persistent severe pain.
   3. Patients with frequent admissions for controlled pain
   4. Patients with drug overdose
The present invention will improve the following:
   1. Patient satisfaction
   2. HCAPS scores,
   3. Significant costs savings,
   4. Improve healthcare professional's employment satisfaction,
   5. Redaction in after-hours phone calls (to doctors, charge nurses, administration),
   6. Reduction in frequent readmissions,
   7. Reduction in harm events, and
   8. Leveraging the acute treatment incidence in the hospital setting as the first step of addressing the Opioid Use Disorder (OUD)

### EXAMPLE

### Representative Inclusion Criteria

A patient's data may be collected for analysis if they meet the diagnosis and main criteria of the analysis as well as any of the following criteria:
Uncontrolled pain on high doses of opioids
PDMP reveals large quantities of opioids or doctor shopping
Evidence of substance abuse (track marks, ETOH intoxication, AMS, frequent falls, prior documentation of “drug seeking”)
Requesting/demanding specific medication, specific route of administration (IVP), specific dose
Allergic to alternative medications other than their drug of choice
Refusal to provide prior medical records
Threatening to leave AMA, sue, call administration if not given what they want

The foregoing descriptions of the embodiments of the present invention have been presented for purposes of illustration and description. They are not intended to be exhaustive or to limit the present invention to the precise forms disclosed. The exemplary embodiments were chosen and described in order to best explain the principles of the present invention and its practical application, to thereby enable others skilled in the art to best utilize the present invention. Although specific embodiments have been illustrated and described herein, a variety of alternate and/or equivalent implementations may be substituted for the specific embodiments shown and described without departing from the scope of the present invention. This application is intended to cover any adaptations or variations of the embodiments discussed herein.

We claim:

1. An algorithm-based system by which potential and current high-risk, opioid dependent patients are assessed and evaluated via a predetermined set of objective and subjective criteria to determine a safe, effective, and consistent mode of treatment that creates a protocol for complex, opioid utilizing and opioid dependent patients in both inpatient and outpatient settings.

2. The algorithm-based system of claim 1, wherein patients are treated uniformly, by all system participating healthcare providers, based on a set number of predetermined measurements where each patient is classified according to need and capability and special attention is paid, in the Initial Encounter, to determine under initial Presentation Diagnosis, Differential Diagnosis, and Early Treatment Factors a patient's designation according the following:
Initial Presentation Diagnostics and Tests
a) Locus or Loci and source of pain (e.g. chronic pain, trauma, post-operation pain, cancer pain, trip and fall, abscess from cellulitis, etc.)
Differential Diagnosis (Establishing Low v. High Risk Patients)
a) History of pain (duration and origin)
Or History of pain relief (non-opioid analgesics, opioid analgesics—high and low dose)
b) Patient claiming multiple drug allergies/specific requests (e.g. specific drugs and routes of administration)
c) Objective evidence of withdrawal
d) Leaving the floor frequently
e) History of controlled substance (e.g. opioid) use
f) History of controlled substance (e.g. opioid) abuse
g) Frequent/multiple hospital admissions
h) History of illicit drug use
i) Evidence of drug use
i. Abscess/cellulitis from IVDA
ii. Claim of spider bite/MRSA
j) Recordation of attempts at detoxifying
k) Evidence of drug (illicit and non-illicit) use through;
Drug/Toxicity screen for drugs and/or alcohol
PDMP (Prescription Drug Monitoring Programs)
Patient requesting a certain route of administration
Patient stating several drug allergies to lower schedule drugs (e.g. tramadol) or non-scheduled drugs (e.g. NSAIDs)
Demanding/Difficult patients
Patient asking for Benadryl®, benzodiazepines (i.e. Valium®, Xanax®, Ativan®), or muscle relaxants (e.g. Soma®) in addition to opioid.
Medical Record Review
Oral History of drug and alcohol consumption
History of certain or particular opioid use (e.g. OxyContin®)
Methadone use
History of large quantity of short acting opioids
Previous DUIs
Early Treatment Factors
a) Contraband search as needed
b) Restriction of visitors as needed
c) Checking PDMP
d) Confirming what narcotic medications patient has at home
e) Call methadone clinic and conform last appointment
f) History of paying cash for medications
g) Accessing whether or not patient has insurance (as this may affect treatment options and substance abuse therapy prospectively)

3. The algorithm-based system of claim 1, wherein patients are treated uniformly, by all system-participating healthcare providers, based on a set number of predetermined measurements and whereas each patient is classified according to need and capability, where special attention is paid, in the Treatment Initiation Phase, to determine under Additional Test and Data, Treatment Efficacy, and Treatment Decisions a patient's designation, according to, and in light of the following:
Additional Test and Data Points
a) Urine Toxicology Screen
b) PDMP (Prescription Drug Monitoring Program(s))
c) ER workup
d) Self-reported opioid use
Treatment Efficacy
a) Measurement of Pain Scorns (via hospital protocol)
b) Recording of vitals
c) Documenting sleep patterns
d) Monitoring disruptive behavior (aggressiveness, demanding behavior, hostile, threatening or intimidating behavior)
e) Monitoring calls to nursing staff and doctor (via nursing staff)
Treatment Decisions (based on Initial Presentation and Differentia Diagnosis)
LOW RISK PATIENTS
a) Opioids/Opiates for low-risk, patients (oral, transdermal, or PCA); or
HIGH RISK PATIENTS
b) Short time opioid PCA or Buprenorphine (Belbuca®, Bunavail®, Buprenex®, Butrans®) and/or buprenorphine/naloxone (Subutex®, Suboxone®)

4. The algorithm-based system of claim 1, wherein patients are treated uniformly, by all system-participating healthcare providers, based on a set number of determined measurements and whereas each patient is classified according to need and capability, where special attention is paid, in the Inpatient Care Phase, to determine, under Additional Test and Data, Treatment Efficacy, and Treatment Decisions a patient's designation according to and, in light of, the following:
Additional Date Tests and Data Points
a) Good pain control—continue therapy
b) Inadequate pain, control—modify therapy
c) Improved NPO status (Nothing by Mouth)—introduce oral (PO) medications including opioids or buprenorphine and/or buprenorphine/naloxone
Treatment Efficacy
a) Assess pain score
b) Assess vital signs for withdrawal
c) Continue monitoring drug screens for possible inconsistency
d) Monitor behaviors and truthfulness of patient
Treatment Decisions
a) Patient well controlled—continue treatment
b) Patient poorly controlled—adjust treatment and consider adjuvant of buprenorphine and/or buprenorphine/naloxone or single buprenorphine and/or buprenorphine/naloxone therapy
c) Identify inconsistences on therapy
d) Continue to monitor pain control

5. The algorithm-based system of claim 1, wherein patients are treated uniformly, by all system-participating healthcare providers, based on a set number of predetermined measurements and whereas each patient is classified according to need and capability, where special attention is paid, in the Discharge Phase, to determine under Additional Test and Data, Risk of Reoccurrence Mitigation, and Treatment Decisions a patient's designation according to, and in light of, the following:
Additional Tests and Data Points
a) Review PDMP to determine the appropriateness of discharge medications
b) Determine presence or absence of insurance coverage to lessen the hurdles to patient access to (1) affordable medication per insurance formulary, (2) affordable treatment post discharge, and (3) substance abuse treatment
Risk of Reoccurrence Mitigation
a) 7-day supply of pain medication is provided at time of discharge with a guaranteed appointment within that 7-day period
b) Referral to substance abuse treatment where appropriate via social services or private concern
Treatment Decisions
a) 7-day supply of medication and guaranteed appoint within that 7-day period
b) Referral to substance abuse treatment facility or private practice (where applicable)

6. The algorithm-based system of claim 1, wherein patients are treated uniformly, by all system-participating healthcare providers, based on a set number of predetermined measurements in the Chronic Management Phase to provide the patient sufficient medication, to treat the patient's pain for 1 week and allowing the patient sufficient time of 1 week to follow up with either the Melrose Pain Solutions® team, via a guaranteed appoint, or other qualified healthcare providers, or to coordinate with hospital staff prior to discharge to attain the proper social services.

7. The algorithm-based system of claim wherein each phase (initial Encounter, Admission, Inpatient Care, and Discharge) have tied to them time, location and staff components that better utilizes limited resources, further enhances the systems' goals of timely and adequate pain control, and facilitates overall better healthcare to patients as follows:
Initial Encounter
a) PCA placement and use initiates faster pain control with less need for nursing intervention
b) Buprenorphine and/or buprenorphine/naloxone delivers pain control for longer periods with less need for nurse intervention
c) Greater communication, a concerted group approach, and single system, utilization between and among healthcare providers provides less redundancy and duplicative action in the Melrose Pain Solution® system
Admission
a) Time location and staff component carries with it the benefit of knowing which steps were taken and at what time In an effort to optimize timely pain control (e.g. within the optimal time of 30 minutes to 6 hours)
Inpatient Care
a) Time location and staff components lead to optimum pain control, increased patient compliance, higher staff satisfaction rates, and higher HCAP scores,
Discharge
a) With a temporal emphasis, the Melrose Pain Solution® system seeks to
i. provide prescription(s) well in advance of patient leaving the facility
ii. recommends filling prescription on-site when possible (beds-to-meds)
iii. deliver substance abuse referrals as appropriate

8. The algorithm-based system of claim 1, which allows for gathering, tracking, sharing, analyzing, utilizing, storing and retrieving of patient information, in a real time and confidential, password secured database, among a health worker peer network to better track and trace potential and current opioid-dependent patients via an computer program or computer application running on a computer system, further wherein the application may be operated using computer hardware, including a computer processor capable of securely safeguarding protected patient information (PPI).

9. The algorithm-based system if claim 1, which allows for tracking and sharing of patient information, in a real time and confidential, password-secured database, among a health worker peer network to better track and trace potential and current opioid-dependent patients via a mobile device (e.g. a mobile application or software application) running on a mobile computer system, further wherein the application may be operated using computer hardware, including a computer processor capable of securely safeguarding protected patient information (PPI).

10. The algorithm-based system of claim 1 that incorporates a means of transmitting and providing password-secured or otherwise selectively available and authorization verifiable access via an internet website where the primary system is parsed into several “subsystems” that allow for a more specific, honed and tailored treatment protocol for each individual patient.

11. The algorithm-based system of claim 1 that incorporates a means of transmitting and providing password-secured or otherwise selectively available and authorization verifiable access via a computer or mobile device and via internet website in use as a tool in providing telemedicine to rural areas.

12. A method for managing and tracking potential and active opioid-dependent patient data for the maintenance and management of treatment of an opioid-utilizing or opioid-dependent pain treatment patient, said method comprising:
establishing clinical criteria and protocol for the assessment, evaluation and placement of patients into categories and sub-categories based on patient presentation, patient history, diagnostic questioning and tests, and a differential diagnosis by a trained healthcare professional upon the initial encounter, in emergency department or urgent care settings, and determining (a) level of pain control, (b) level of opioid use, (c) level of opioid dependency, and (d) clinical stability;
placing patient into one of two categories (a) outpatient or (b) inpatient (hospital admitted);
treating outpatient individuals with (a) 3 to 7-day supply of opioids with pain physician referral for continued care or (2), after verifying opioid abuse through a state controlled substances monitoring program (e.g. PDMP—Prescription Drug Monitoring Program) and Integration of additional considerations as to opioid usage, dispensing non-opioid medications or (3) dispensing buprenorphine and/or buprenorphine/naloxone with referral to a detoxification center or dependency drug authorized dispenser;
treating inpatient (hospitalized) patients, through additional testing, varying data points determinations, and treatment decisions, with (1) continued patient controlled analgesia pump, oral or transdermal opioid with or without a non-opioid analgesic, (2) acute buprenorphine and/or buprenorphine/naloxone with a transition to a maintenance buprenorphine and/or buprenorphine/naloxone, or (3) short acting opioids with a transition to maintenance buprenorphine and/or buprenorphine/naloxone;
discharging patients after observation and subsequent stabilization with an establishment of risk/likelihood of reoccurrence and outpatient treatment plan (taking into consideration possible suboptimal pain therapy regimen, ineffective therapies, inability to take certain formulations, long-acting versus short-acting opioids, and history of substance abuse) up to and including modification of current drug treatment and possible inclusion of buprenorphine and/or buprenorphine/naloxone.

13. The method according to claim 12 wherein a plurality of program possibilities are established, and one direction of treatment best suiting the patient's needs and abilities is selected based on a set protocol for complex, opioid utilizing and opioid dependent patients in both inpatient and outpatient settings.

14. The method described in claim 12 wherein a plurality of program possibilities are established, and one direction of treatment best suiting the patient's needs and abilities is selected, at time of initial assessment, based on a set protocol for complex, opioid utilizing and opioid dependent patients (in both inpatient and outpatient settings) where revaluation and reassessment, at some time alter initial assessment, reveals the necessity to restructure and reimplement an evolved and revised treatment plan resulting in a new treatment direction.

15. The method of claim 12, further compromising:
providing a moans to track, trace, store, analyze, retrieve and electronically display the number of prescriptions (and number of individual dosages) received by a specific patient;
providing a means to track, trace, store, analyze, retrieve and electronically display the number of emergency department or urgent care settings visited by a specific patient in a given period of time;
providing a means to track, trace, store analyze, retrieve and electronically display the number of prescribers prescribing for a specific patient in a set or variable period of time;
displaying, in a graphical display, via a computer interface, application or mobile application, a graphic representation of prescriptions and unit dosage received, the number of prescribers prescribing, and the number of urgent care and ER visits of a specific patient.

16. The method of claim 12, further compromising:
providing a means for determining the risk or level of opioid use or activity of an individual during set or variable periods of time and storing said information regarding the risk or level of opioid use;
calculating and analyzing the cumulative opioid intake or morphine equivalent intake for the period of time;
storing, retrieving, and displaying, in a graphical display, via a computer interface, application or mobile application, a comparison of the opioid usage and intake for the period of time.

17. The method according to claim 12, wherein a plurality of programs are established and one best suiting the patient's needs or capabilities is selected and possibly reselected based on changing patient variables and revaluations and reassessments based on such variables in progression to a new treatment plan.

18. The method of claim 12, further compromising:
an interactive system that may optionally include one or more audio inputs (e.g. microphones) and outputs (e.g. speakers) and/or video capabilities, and accompanying hardware and software components that allow a user to listen to audible components from a file (on a computer, web interface, mobile application, or a combination of the three) and/or watch recorded content, where the interactive system, may also optionally include a microphone and accompanying hardware and/or software audio visual components that allow a user to record his or hex own audio and video input which, for instance, can be transcribed stored allowing a healthcare practitioner to contribute additional information, e.g., regarding a patient's status.

19. The method of claim 12, further compromising:
the capability to mathematically sort, stare, categorize, classify, and present information in the aggregate, without referral to a specific patient, to further the understanding of opioid dependency and to allow for greater insight and understanding of opioid abuse, resulting in more effective, adaptive future treatment choices and decisions;
the ability to display cumulative data, numerically, graphically, or in such a manner the statistical data accumulated can be better interpreted or understood by clinicians, practitioners, and statisticians.
